SEQUENCE LISTING (11 sequences)

SEQ ID NO: 1 - protein, 711 aa, Homo sapiens [sequence omitted]

SEQ ID NO: 2 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 3 - protein, 708 aa, Ovis aries
MKLFVPALLSLGALGLCLAAPRKNVRWCAISPPEGSKCYQWQRRMRKLGAPSITCVRRTSALECIRAIAGKKADAVTLDSGMVFEAGLDPYKLRPVAAEIYGTEKSPQTHYYAVAVVKKGSNFQLDQLQGQKSCHMGLGRSAGWNIPMGILRPFLSWTESAEPLQGAVARFFSASCVPCVDGKAYPNLCQLCKGVGENKCACSSQEPYFGYSGAFKCLQDGAGDVAFVKETTVFENLPEKADRDQYELLCLNNTRAPVDAFKECHLAQVPSHAVVARSVDGKENLIWELLRKAQEKFGKNKSQRFQLFGSPQGQKDLLFKDSALGFVRIPSKVDSALYLGSRYLTALKNLRETAEEVKARCTRVVWCAVGPEEHSKCQQWSEQSGQNVTCAMASTTDDCIALVLKGEADALSLDGGYIYTAGKCGLVPVMAENRESSKYSSLDCVLRPTEGYLAVAVVKKANEGLTWNSLKGKKSCHTAVDRTAGWNIPMGLIANQTGSCAFDEFFSQSCAPGADPKSSLCALCAGDDQGLNKCVPNSKEKYYGYTGAFRCLAEDVGDVAFVKNDTVWENTNGESSADWAKNLNREDFRLLCLDGTTKPVTEAQSCYLAVAPNHAVVSRSDRAAHVEQVLLHQQALFGKNGKNCPDQFCLFKSETKNLLFNDNTECLAKLGGRPTYEKYLGTEYVTAIANLKKCSTSPLLEACAFLTR

SEQ ID NO: 4 - protein, 708 aa, Capra hircus
MKLFVPALLSLGALGLCLAAPRKNVRWCAISLPEWSKCYQWQRRMRKLGAPSITCIRRTSALECIRAIAGKNADAVTLDSGMVFEAGLDPYKLRPVAAEIYGTEKSPQTHYYAVAVVKKGSNFQLDQLQGQKSCHMGLGRSAGWNIPVGILRPFLSWTESAEPLQGAVARFFSASCVPCVDGKAYPNLCQLCKGVGENKCACSSQEPYFGYSGAFKCLQDGAGDVAFVKETTVFENLPEKADRDQYELLCLNNTRAPVDAFKECHLAQVPSHAVVARSVDGKENLIWELLRKAQEKFGKNKSQSFQLFGSPEGRRDLLFKDSALGFVRIPSKVDSALYLGSRYLTALKNLRETAEELKARCTRVVWCAVGPEEQSKCQQWSEQSGQNVTCATASTTDDCIALVLKGEADALSLDGGYIYTAGKCGLVPVMAENRKSSKYSSLDCVLRPTEGYLAVAVVKKANEGLTWNSLKGKKSCHTAVDRTAGWNIPMGLIANQTGSCAFDEFFSQSCAPGADPKSSLCALCAGDDQGLDKCVPNSKEKYYGYTGAFRCLAEDVGDVAFVKNDTVWENTNGESSADWAKNLNREDFRLLCLDGTTKPVTEAQSCYLAVAPNHAVVSRSDRAAHVEQVLLHQQALFGKNGKNCPDQFCLFKSETKNLLFNDNTECLAKLGGRPTYEKYLGTEYVTAIANLKKCSTSPLLEACAFLTR

SEQ ID NO: 5 - protein, 708 aa, Equus caballus
MKLLFPVLLSLGALGLCLAAPRKSVRWCTISPAEAAKCAKFQRNMKKVRGPSVSCIRKTSSFECIQAIAANKADAVTLDGGLVYEAGLHPYKLRPVAAEVYQTRGKPQTRYYAVAVVKKGSGFQLNQLQGVKSCHTGLGRSAGWNIPIGTLRPYLNWTGPPEPLQKAVANFFSASCVPCADGKQYPNLCRLCAGTEADKCACSSQEPYFGYSGAFKCLENGAGDVAFVKDSTVFENLPDEADRDKYELLCPDNTRKPVDAFKECHLARVPSHAVVARSVDGREDLIWRLLHRAQEEFGRNKSSAFQLFKSTPENKDLLFKDSALGFVRIPSQIDSGLYLGANYLTATQNLRETAAEVAARRERVVWCAVGPEEERKCKQWSDVSNRKVACASASTTEECIALVLKGEADALNLDGGFIYVAGKCGLVPVLAENQKSQNSNAPDCVHRPPEGYLAVAVVRKSDADLTWNSLSGKKSCHTGVGRTAGWNIPMGLLFNQTGSCKFDKFFSQSCAPGADPQSSLCALCVGNNENENKCMPNSEERYYGYTGAFRCLAEKAGDVAFVKDVTVLQNTDGKNSEPWAKDLKQEDFELLCLDGTRKPVAEAESCHLARAPNHAVVSQSDRAQHLKKVLFLQQDQFGGNGPDCPGKFCLFKSETKNLLFNDNTECLAELQGKTTYEQYLGSEYVTSITNLRRCSSSPLLEACAFLRA

SEQ ID NO: 6 - protein, 708 aa, Camelus dromedarius
MKLFFPALLSLGALGLCLAASKKSVRWCTTSPAESSKCAQWQRRMKKVRGPSVTCVKKTSRFECIQAISTEKADAVTLDGGLVYDAGLDPYKLRPIAAEVYGTENNPQTHYYAVAIAKKGTNFQLNQLQGLKSCHTGLGRSAGWNIPMGLLRPFLDWTGPPEPLQKAVAKFFSASCVPCVDGKEYPNLCQLCAGTGENKCACSSQEPYFGYSGAFKCLQDGAGDVAFVKDSTVFESLPAKADRDQYELLCPNNTRKPVDASQECHLARVPSHAVVARSVNGKEDLIWKLLVKAQEKFGRGKPSAFQLFGSPAGQKDLLFKDSALGLLRIPSKIDSGLYLGSNYITAIRGLRETAAEVELRRAQVVWCAVGSDEQLKCQEWSRQSNQSVVCATASTTEDCIALVLKGEADALSLDGGYIYIAGKCGLVPVLAESQQSPESSGLDCVHRPVKGYLAVAVVRKANDKITWNSLRGKKSCHTAVDRTAGWNIPMGPLFKNTDSCRFDEFFSQSCAPGSDPRSKLCALCAGNEEGQNKCVPNSSERYYGYTGAFRCLAENVGDVAFVKDVTVLDNTDGKNTEQWAKDLKLGDFELLCLNGTRKPVTEAESCHLPVAPNHAVVSRIDKVAHLEQVLLRQQAHFGRNGQDCPGKFCLFQSKTKNLLFNDNTECLAKLQGKTTYEEYLGPQYVTAIAKLRRCSTSPLLEACAFLMR

SEQ ID NO: 7 - protein, 11 aa, synthetic construct
  REGION 1..11: note = hLfcin1-11 (hLf peptide)
GRRRRSVQWCA

SEQ ID NO: 8 - protein, 351 aa, Homo sapiens
MKLVFLVLLFLGALGLCLAGRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNL

SEQ ID NO: 9 - protein, 930 aa, synthetic construct
  REGION 1..930: note = hLF-CH2-CH3 fusion protein
MKLVFLVLLFLGALGLCLAGRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRKDPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 1298 aa, synthetic construct
  REGION 1..1298: note = hLF-HSA fusion protein
MKLVFLVLLFLGALGLCLAGRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRKDPDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 11 - protein, 890 aa, synthetic construct
  REGION 1..890: note = hLF-G-CSF fusion protein
MKLVFLVLLFLGALGLCLAGRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRKSRLEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2025/0025537 A1
(43) Pub. Date: Jan. 23, 2025

(54) GLYCOSAMINOGLYCAN INHIBITOR AND PROMOTER

(71) Applicant: S&K BIOPHARMA, INC., Kanagawa (JP)

(72) Inventors: Masao NAKAMURA, Tokyo (JP); Atsushi SATO, Kanagawa (JP); Shinji KAGAYA, Kanagawa (JP)

(21) Appl. No.: 18/883,966

(22) Filed: Sep. 12, 2024

Related U.S. Application Data
(63) Continuation of application No. 17/125,658, filed on Dec. 17, 2020, now abandoned.
Application No. 17/125,658 is a continuation-in-part of application No. PCT/JP2019/025118, filed on Jun. 25, 2019.

(30) Foreign Application Priority Data
Jun. 26, 2018 (JP) 2018-121377

(51) Int. Cl. A61K 38/40; C12N 9/88

(52) U.S. Cl. CPC A61K 38/40; C12N 9/88; C12Y 402/0202

(57) ABSTRACT

The purpose of the present invention is to develop a medicine for diseases related to glycosaminoglycan functions with less side effects. Provided is a glycosaminoglycan inhibitor or promoter that comprises lactoferrin or a derivative thereof.

### CROSS-REFERENCE TO RELATED APPLICATIONS

The present application is a continuation of U.S. application Ser. No. 17/125,658, filed on Dec. 17, 2020, which is a continuation-in-part of International Application No. PCT/JP2019/025118, filed on Jun. 25, 2019, which claims benefit of priority to Japanese Patent Application No. 2018-121377, filed on Jun. 26, 2018, the entire contents of which applications are incorporated herein by reference.

### INCORPORATION BY REFERENCE OF SEQUENCE LISTING PROVIDED AS A SEQUENCE LISTING XML FILE

A Sequence Listing is provided herewith as a Sequence Listing XML, “G2086US-CON1.xml”, created on Sep. 12, 2024, and having a size of 19,659 bytes. The contents of the Sequence Listing XML are incorporated herein by reference in their entirety.

### TECHNICAL FIELD

The present invention relates to inhibitors and promoters of glycosaminoglycans. More specifically, the present invention relates to an inhibitor or promoter of glycosaminoglycan comprising lactoferrin or a derivative thereof as an active ingredient, and a medical use of lactoferrin in the treatment of glycosaminoglycan-related diseases based on the glycosaminoglycan inhibitory or promotive action of lactoferrin.

### BACKGROUND ART

Most glycosaminoglycans are in a form of proteoglycans, in which polysaccharides are added to a protein called a core protein, present in the extracellular matrix and cell surfaces of body tissues, and present as a major component of cartilage. Among others, chondroitin sulfate interacts with various cell growth factors and extracellular matrix components to regulate various cellular activities such as cell adhesion, migration, proliferation, differentiation, and morphogenesis. Moreover, medicines and food products made from sodium chondroitin sulfate are available on the market for the treatment of low back pain, joint pain, peri-articular inflammation of the shoulder, and so on. In the following, abbreviations are indicated as follows concerning sugars: chondroitin sulfate A (CS-A), chondroitin sulfate B (CS-B), chondroitin sulfate C (CS-C), chondroitin sulfate D (CS-D), chondroitin sulfate E (CS-E), hyaluronic acid (HA), heparin (HP), heparan sulfate (HS), and keratan sulfate (KS).

Excessive expression of chondroitin sulfate in inflammatory areas is associated with spinal cord injury, cancer metastasis, and Alzheimer's disease, leading to worsening of the disease. It is estimated that more than 100,000 people in Japan already suffer from spinal cord injuries in which chondroitin sulfate is involved in the improvement of the disease, with approximately 5,000 new cases each year, and approximately 200,000 new cases worldwide. The disease can be classified into an acute phase from the time of injury up to a few hours, a subacute phase up to a few weeks, and a chronic phase thereafter. While some degree of tissue repair and spontaneous recovery is achieved up to the subacute phase, after the chronic phase, the reconstruction of disrupted neural circuits is inhibited and recovery is difficult due to glial scars that have formed tightly at the site of injury (Non-Patent Document 1). Currently, recovery with high doses of steroids, hepatocyte growth factors, and regenerative medicine within 72 hours of injury has been tried, but no treatment has been established.

In spinal cord injury, reactive astrocytes, a major factor in glial scarring that inhibits the reconstruction of neural circuits, synthesize chondroitin sulfate proteoglycans.

The receptors for glycosaminoglycans, PTPσ and LARs, are present on the surface of the growth cones of neurons at the site of injury, and it has been reported that chondroitin sulfate proteoglycans, especially CS-E glycans, are involved in the inhibition of axonal regeneration by binding to these receptors (Non-Patent Document 2). Therefore, in experiments using a spinal cord injury model, therapeutic effects have been reported with enhanced nerve regeneration by PTPσ of the CS-E receptor (Non-Patent Document 3) and knockout mice of the phosphatase LAR (Non-Patent Document 4), degradation of CS-E by the bacterial enzyme chondroitinase ABC, which degrades glycans that bind to the proteoglycan core protein (Non-Patent Documents 5, 6) and axonal regeneration by CS-E synthase inhibitors (Non-Patent Document 7).

In recent years, a new treatment method using chondroitinase ABC has been marketed for the treatment of lumbar disc herniation, with the promise of topical administration. However, because this product is a bacterial protein, side effects (including abnormalities in laboratory test results) were observed in 53.3% of cases in domestic clinical trials. The most common were Modic classification vertebral luminosity changes (23.6%), low back pain (22.3%), disc height reduction of more than 30% (14.4%), and lower extremity pain (4.8%), with the most serious ones being shock and anaphylaxis reported in the study. Thus, it is essential to develop drugs with fewer side effects.

In humans, there is an enzyme called hyaluronidase that degrades chondroitin sulfate. Hyaluronidase is not effective when administered to a dog model of spinal cord injury (Non-Patent Document 8), and when administered to a mouse model of spinal cord injury, no improvement in locomotion has been observed, but rather a tendency to worsen inflammation has been observed (Non-Patent Document 9). Treatment as regenerative medicine is also being considered, and in 2018, treatment with cultured autologous bone marrow mesenchymal stem cells has been approved. Attempts to regenerate damaged neurons from iPS cells have also been considered, but regenerative medicine has the disadvantage of being expensive to treat and taking a long time before the cells can be administered, making it impossible to respond to treatment at an early stage after an injury.

It has also been reported that chondroitin sulfate is highly expressed on the surface of cancer cells and has been implicated in cancer metastasis (Non-Patent Document 10). In particular, in lung cancer, cancer cells have been found to metastasize when RAGE proteins expressed in cells of normal lung tissue, especially in the vascular endothelium, bind to CS-E and other proteins (Non-Patent Documents 11 and 12). That metastasis is inhibited by chondroitinase ABC, anti-CS-E, and anti-RAGE antibodies. Glycosaminoglycans such as hyaluronic acid and CS-C have also been reported to bind to CD44, a surface marker of breast cancer stem cells involved in breast cancer metastasis (Non-Patent Document 10).

Lactoferrin (Lf), on the other hand, is an iron-binding glycoprotein with a molecular weight of about 80,000 that belongs to the transferrin family and is abundantly present in mammalian exocrine fluids such as colostrum and tears. Lactoferrin is composed of globular domains of N-lobe and C-lobe, and one trivalent iron is bound to each domain. The conformation is different between the iron-saturated (Holo) and iron-free (Apo) forms. The three-dimensional structure of human lactoferrin is shown in FIG. 10.

Lactoferrin is a cell-secreted molecule that bridges innate and adaptive immune functions in mammals, and its protective effects range from anti-cancer, anti-inflammatory, and immunomodulatory activities to antimicrobial activity against many microorganisms. This wide range of activities is reported to be made possible not only by the iron-binding capacity of Lf but also by a mechanism of action involving the interaction of Lf with molecular and cellular components of the host and pathogen (Non-Patent Document 13).

### BACKGROUND ART DOCUMENTS

### Non-Patent Documents

Non-Patent Document 1: J. Neurotrauma 2005; 22 (5), 544-558
Non-Patent Document 2: Trends in Glycoscience and Glycotechnology 2011; 23 (133) 201-211
Non-Patent Document 3: Science 2009 Oct. 23; 326 (5952): 592-6
Non-Patent Document 4: Neurobiol Dis 2015; 73:36-48
Non-Patent Document 5: FEBS Journal 280 (2013) 2462-2470
Non-Patent Document 6: Nature. 2002 Apr. 11; 416 (6881): 636-40
Non-Patent Document 7: ACS Chem. Biol. 2017, 12, 3126-3133
Non-Patent Document 8: Surg. Neurol. 13, 157-159, 1980
Non-Patent Bibliography 9: Proceedings of the 37th Annual Meeting of the Japanese Society of Carbohydrate Science, Oda et al. Aug. 30, 2018
Non-Patent Document 10: Biochemical Journal (2018) 475 587-620
Non-Patent Document 11: THE JOURNAL OF BIOLOGICAL CHEMISTRY 2008; 283 (49): 34294-34304
Non-Patent Document 12: Biomed Res Int. 2013:656319
Non-Patent Document 13: Biochimica et Biophysica Acta, Volume 1820, Issue 3, Mar. 2012, Pages 226-236

### SUMMARY OF THE INVENTION

Therefore, there is a need for the development of medicines with fewer side effects for diseases related to the function of glycosaminoglycans.

As a means of overcoming the above shortcomings, the inventors propose a pharmaceutical composition with minimal side effects for diseases related to the function of glycosaminoglycans, typically using a safe human lactoferrin (hLF) and its functional moiety, derivatives, and the like, and a method of treating said diseases using the same.

In the search for biological substances that inhibit or enhance the function of glycosaminoglycans involved in the exacerbation of multiple diseases, the inventors have focused on lactoferrin and have examined whether lactoferrin inhibits or enhances the function of certain glycosaminoglycans. Using lactoferrin, its N-lobe, and the basic peptides contained in lactoferrin, the inventors have found that only certain glycosaminoglycans bind to lactoferrin by circular dichroism (CD) spectra, that pharmacological activity in ameliorating spinal cord injury is based on the axonal elongation activity of lactoferrin using a cellular model, and the cancer therapeutic effects include inhibition of cancer cell line growth, rate of cell death, and inhibition of cancer cell invasion and migration capacity by scratch assays. Based on these findings, they found that lactoferrin, and peptides, globular domains, mutants, and derivatives thereof having its activity could be effective therapeutic agents for diseases against certain glycosaminoglycans, and the present invention was completed.

The present disclosure includes the following [1] to [19].
[1] An inhibitor or promoter composition of glycosaminoglycans comprising lactoferrin or derivatives thereof.
[2] The inhibitor or promoter composition according to [1], wherein the lactoferrin is an iron-free (Apo) or iron saturated (Holo) lactoferrin.
[3] The inhibitor or promoter composition according to [1] or [2], wherein the lactoferrin is of human origin.
[4] The inhibitor or promoter composition according to [1], wherein the lactoferrin is a protein or peptide selected from the group consisting of (a) to (e) below.
   (a) a protein consisting of any one amino acid sequence of SEQ ID NOS: 1-6;
   (b) a protein comprising N-lobe of lactoferrin;
   (c) a protein consisting of the amino acid sequence in which 1 to 70 amino acids are deleted, substituted, inserted, and/or added in any one amino acid sequence of SEQ ID NOS: 1 to 6, and which has the activity to inhibit or promote the expression or function of chondroitin sulfate; and
   (d) a protein consisting of the amino acid sequence having 90% or more sequence identity to any one amino acid sequence of SEQ ID NOS: 1-6 and having activity that inhibits or promotes the expression or function of chondroitin sulfate; and
   (e) a peptide consisting of the amino acid sequence of GRRRRSVQWCA (SEQ ID NO: 7).
[5] The inhibitor or promoter composition according to any one of [1] to [4], the derivative of the lactoferrin is a hinge deletion human lactoferrin/human IgG Fc fusion protein (hLF-CH2-CH3) (SEQ ID NO: 9), human lactoferrin/human serum albumin fusion protein (hLF(-HSA) (SEQ ID NO: 10) or human lactoferrin-human granulocyte colony-stimulating factor (G-CSF) fusion protein (hLF-G-CSF) (SEQ ID NO: 11).
[6] The inhibitor or promoter composition according to any one of [1] to [5], wherein the glycosaminoglycan is any one selected from the group consisting of chondroitin sulfate-A (CS-A), chondroitin sulfate-B (CS-B), chondroitin sulfate-C (CS-C), chondroitin sulfate-D (CS-D), chondroitin sulfate-E (CS-E), heparin (HP) and heparan sulfate (HS), and keratan sulfate (KS).
[7] The inhibitor or promoter composition according to [6], wherein the glycosaminoglycan is chondroitin sulfate-C (CS-C), chondroitin sulfate-D (CS-D), or chondroitin sulfate-E (CS-E).
[8] The inhibitor or promoter composition according to [6], wherein the glycosaminoglycan is CS-E.
[9] A pharmaceutical composition for treating a glycosaminoglycan-related disease or condition, comprising lactoferrin or a derivative thereof.
[10] The pharmaceutical composition according to [9], wherein the lactoferrin is iron-free (Apo) or iron saturated (Holo) lactoferrin, prepared in an amount from 0.001 to 10 g/kg/day.
[11] The pharmaceutical composition according to [9] or [10], wherein the lactoferrin is of human origin.
[12] The pharmaceutical composition according to [9], wherein the lactoferrin is a protein or peptide selected from the group consisting of (a) to (e) below:
   (a) a protein consisting of any one amino acid sequence of SEQ ID NOS: 1-6;
   (b) a protein comprising N-lobe of lactoferrin;
   (c) a protein consisting of the amino acid sequence in which 1 to 70 amino acids are deleted, substituted, inserted, and/or added in any one amino acid sequence of SEQ ID NOS: 1 to 6, and which has the activity to inhibit or promote the expression or function of glycosaminoglycans; and
   (d) a protein consisting of the amino acid sequence having 90% or more sequence identity to any one amino acid sequence of SEQ ID NOS: 1-6 and having activity that inhibits or promotes the expression or function of chondroitin sulfate; and
   (e) a peptide consisting of the amino acid sequence of GRRRRSVQWCA (SEQ ID NO: 7).
[13] The pharmaceutical composition according to any one of [9] to [12], wherein the derivative of the lactoferrin is a hinge deletion human lactoferrin/human IgG Fc fusion protein (hLF-CH2-CH3) (SEQ ID NO: 9), human lactoferrin/human serum albumin fusion protein (hLF-HSA) (SEQ ID NO: 10) or human lactoferrin-human granulocyte colony-stimulating factor (G-CSF) fusion protein (hLF-G-CSF) (SEQ ID NO: 11).
[14] The pharmaceutical composition according to any one of [9] to [13], wherein the glycosaminoglycan is chondroitin sulfate-C (CS-C), chondroitin sulfate-D (CS-D), or chondroitin sulfate-E (CS-E).
[15] The pharmaceutical composition according to any one of [9] to [14], for use in combination with chondroitin sulfate-degrading enzymes.
[16] The pharmaceutical composition according to any one of [9] to [15], wherein the disease is any one selected from the group consisting of spinal cord injury, cancer metastasis, cancer, diabetes, inflammation, atherosclerosis, multiple sclerosis, fibrosis, acute respiratory disease, and Alzheimer's disease.
[17] The pharmaceutical composition according to any one of [9] to [16], which is in the form of a food product.
[18] The pharmaceutical composition according to any one of the [9] to [16], which is in the form of an injection.
[19] The pharmaceutical composition according to any one of [9] to [16], wherein the pharmaceutical composition is for an oral administration.

The inventors have found that hLF, hLF N-lobe, and their derivatives, hLF-CH2-CH3, hLF-HSA, hLF-G-CSF bind strongly to CS-E, a glycosaminoglycan. In experiments with chick embryo spinal dorsal root ganglion neurons, CS-E inhibition of nerve axon outgrowth and disruption of growth cones were observed, while it was observed that the addition of hLF, hLF N-lobe, hLF-CH2-CH3, hLF-HSA, and hLF-G-CSF resulted in inhibition of axonal elongation and protection against disruption of growth cones. This indicated their potential for therapeutic applications, especially in the acute phase.

A glycosaminoglycan inhibitor/promoter composition containing lactoferrin or a derivative thereof is useful as a medicine for the treatment of diseases or conditions related to the function of glycosaminoglycans.

### BRIEF DESCRIPTION OF THE DRAWINGS

FIG. 1 shows the results of secondary structure analysis of (Apo and Holo) full-length human lactoferrin (hLf) with different degrees of iron-binding in the presence of various glycosaminoglycans by CD spectroscopy. (A) Results of secondary structure analysis of iron-saturated hLf (Holo hLf): (B) Results of secondary structure analysis of iron-free hLf (Apo hLf).

FIG. 2 shows the results of secondary structure analysis of hLf N-lobe with different degrees of iron-binding in the presence of various glycosaminoglycans by CD spectroscopy. (A) Secondary structure analysis of the iron-saturated hLf N-lobe (Holo hLf N-lobe): (B) Secondary structure analysis of the iron-free hLf N-lobe (Apo hLf N-lobe).

FIG. 3 shows the results of thermal denaturation studies of full-length human lactoferrin (hLf) and hLf N-lobe with different degrees of iron-binding in the presence of various glycosaminoglycans, respectively, by CD spectroscopy. (A) Thermal stability of Apo/Holo hLf (left) and Apo/Holo hLf N-lobe (right) in the absence of glycosaminoglycans is shown. (B) Thermal stability of Apo hLf in the presence of various glycosaminoglycans (CS-A to E (left), HP, and HS (right)). (C) Thermal stability of Holo hLf N-lobe in the presence of various glycosaminoglycans (CS-A to E (left), HP, and HS (right)). (D) Thermal stability of Apo hLf N-lobe in the presence of various glycosaminoglycans (CS-A to E (left), HP, and HS (right)).

FIG. 4 shows the results of secondary structure analysis of hLfcin1-11 (GRRRRSVQWCA (SEQ ID NO: 7)) in the presence of various glycosaminoglycans by CD spectroscopy.

FIG. 5 is a graph showing the effect of hLF N-lobe on cell proliferation of chlorate-treated (NaClO₃)-treated non-small cell lung cancer cell line PC-3 in the presence of various glycosaminoglycans (CS-A to E).

FIG. 6 is a diagram showing the effect of iron-saturated (Holo) hLF N-lobe on cell proliferation of (A) chlorate (NaClO₃) or (B) chondroitin sulfate-degrading enzyme (ChABC)-treated non-small cell lung cancer cell line PC-3.

FIG. 7 is a diagram showing the effect of hLF N-lobe on CS-E-dependent metastasis of human lung cancer PC-14 cells.

FIG. 8 is a diagram showing the effect of various glycosaminoglycans (CS-A to E) on protrusion stretching and growth cones of PC-12 cells.

FIG. 9 is a diagram showing the effects of hLf N-lobe on the protrusion elongation and growth cones of PC-12 cells in the presence of various glycosaminoglycans (CS-A to E).

FIG. 10 is a diagram showing the three-dimensional structure of human lactoferrin (background art).

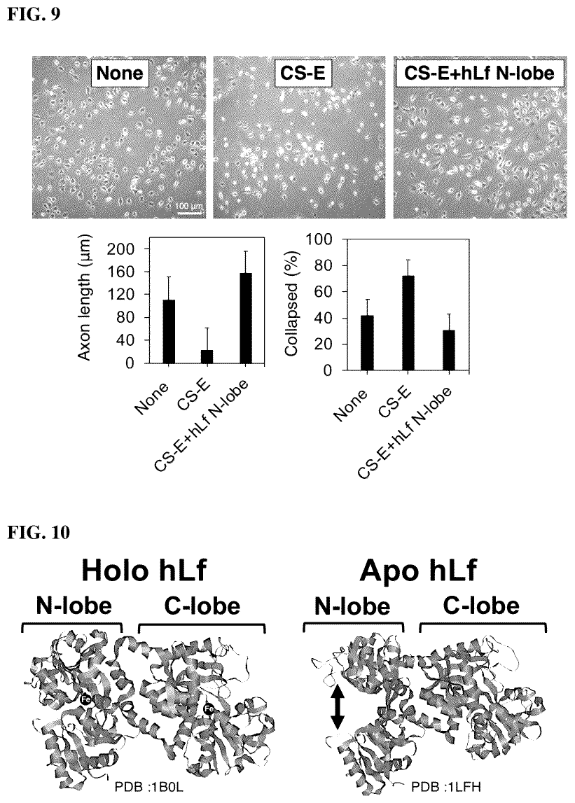

FIG. 11 is a diagram showing the results of binding analysis of human lactoferrin (hLF), hLF N-lobe, and hLF derivatives (hLF-CH2-CH3, hLF-HSA, hLF-G-CSF) with various sulfated glycosaminoglycans (sGAGs) by solid-phase binding assay.

FIG. 12 is a diagram showing the inhibitory effects of hLF, hLF N-lobe, and hLF derivatives (hLF-CH2-CH3, hLF-HSA, and hLF-G-CSF) on the axonal outgrowth inhibition activity of CS-E in chick embryo spinal cord dorsal root ganglion (DRG) cells.

FIG. 13 is a diagram showing the results of the inhibitory effects of hLF, hLF N-lobe, and hLF derivatives (hLF-CH2-CH3, hLF-HSA, hLF-G-CSF) on the growth cone collapse activity of CS-E chick embryo spinal cord DRG cells.

### EMBODIMENTS FOR IMPLEMENTING THE INVENTION

### 1. Definition of Terms

The term “lactoferrin” as used herein means full-length lactoferrin, a protein consisting of a functional moiety of lactoferrin, or a protein containing such a functional moiety, unless otherwise stated. As used herein, “functional moiety” of lactoferrin means a portion of lactoferrin or a variant(s) of lactoferrin that by itself has a function equivalent to or at least partly equivalent to that of full-length lactoferrin. Full-length lactoferrin includes but are not limited to human lactoferrin (SEQ ID NO: 1), bovine lactoferrin (SEQ ID NO: 2), sheep lactoferrin (SEQ ID NO: 3), goat lactoferrin (SEQ ID NO: 4), horse lactoferrin (SEQ ID NO: 5), and camel lactoferrin (SEQ ID NO: 6) and others.

Information on the amino acid sequences of these lactoferrins and the nucleotide sequences encoding them is available by accessing sequence databases (e.g., the NCBI database) that are readily accessible on the Web. For convenience, Table 1 below shows these human and some mammalian sequence information.

TABLE 1

| Origin of lactoferrin | Genbank Accession No. | SEQ ID NO: |
|---|---|---|
| Human | ANC33015.1 | 1 |
| Bovine | NP_851341.1 | 2 |
| Sheep | ACT76166.1 | 3 |
| Goat | AAA97958.1 | 4 |
| Horse | NP_001157446.1 | 5 |
| Camel | AAF82241.1 | 6 |

The term “function of lactoferrin” herein means the ability to inhibit or promote the expression or function of a glycosaminoglycan, at least in vitro and preferably in vivo. The “function of the glycosaminoglycan” includes any function of the glycosaminoglycan that is recognized by those skilled in the art as a function of the glycosaminoglycan, including, but not limited to, the regulation of various cellular activities such as cell adhesion, migration, proliferation, differentiation, and morphogenesis.

The “functional part” of lactoferrin typically includes
(1) the globular domain of lactoferrin (N-lobe or C-lobe);
(2) lactoferrin variants (e.g., a protein consisting of an amino acid sequence with 90% or more identity to any of the amino acid sequences of SEQ ID NOS: 1-6, or an amino acid sequence having substitutions, deletions, insertions, and/or additions of one to several amino acid residues (e.g., from two to about 70) in any of the amino acid sequences of SEQ ID NOS: 1-6);
(3) a partial peptide of lactoferrin (e.g., a peptide consisting of amino acid residues 20-30 of human lactoferrin (“GRRRRSVQWCA” (SEQ ID NO: 7)); and the like.

The variants mentioned in (2) above are typically, but not limited to, variants of lactoferrin having any one amino acid sequence of naturally occurring SEQ ID NOS: 1-6, and include those that can be acquired artificially using site-specific mutagenesis methods described in, for example, Manual Vol. 3, Cold Spring Harbor Laboratory Press 2001″, “Ausubel, Current Protocols in Molecular Biology, John Wiley & Sons 1987-1997”, “Nuc. Acids. Res., 10, 6487 (1982)”, “Proc. Natl. Acad. Sci. USA, 79, 6409 (1982)”, “Gene, 34, 315 (1985)”, “Nuc. Acids. Res., 13, 4431 (1985)”, “Proc. Natl. Acad. USA, 82, 488 (1985)” and others.

In the example of the variants of (2) above, the degree of mutation by substitutions, deletions, insertions, and/or additions of amino acid residues in the amino acid sequence is permissible as long as the variant protein has the same or at least some of the functions of the original protein, and the degree can be typically up to about 10% of the original amino acid sequence, i.e., from one to several (e.g., from two to about 70 sequentially) in the number of amino acid residues.

In the example of the variants in (2) above, “90% or more identity” may include 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, and 99.9% or more sequence identity to the original amino acid sequence.

As used herein, “derivative(s) of lactoferrin” means a lactoferrin molecule with a desired modification to lactoferrin by genetic engineering techniques. Examples of such a derivative(s) of lactoferrin include, for example, a fusion protein of lactoferrin with another protein. Examples of such fusion proteins include a hinge deletion human lactoferrin/human IgG Fc fusion protein (hLF-CH2-CH3) (SEQ ID NO: 9), human lactoferrin/Human serum albumin fusion protein (hLF-HSA) (SEQ ID NO: 10), human lactoferrin-human granulocyte colony-stimulating factor (G-CSF) fusion protein (hLF-G-CSF) (SEQ ID NO: 11), etc.

For lactoferrin, “ability to inhibit or promote the expression or function of glycosaminoglycans” can be quantitatively measured by comparing measures of glycosaminoglycan expression or function in the absence and presence of lactoferrin, and verifying that the above measures are reduced or increased in the presence of lactoferrin. Non-limiting examples of such quantitative measurement methods are shown in the examples described later in this specification.

As used herein, “glycosaminoglycan” is used in the sense generally recognized by those skilled in the art and refers to a long non-branched polysaccharide comprising a repeating disaccharide unit. Examples of glycosaminoglycans concerning the present invention include but are not limited to chondroitin sulfate A (CS-A), chondroitin sulfate B (CS-B), chondroitin sulfate C (CS-C), chondroitin sulfate D (CS-D), chondroitin sulfate E (CS-E), hyaluronic acid (HA), heparin (HP), heparan sulfate (HS), keratan sulfate (KS), etc.

### 2. Glycosaminoglycan Inhibitors or Accelerators Containing Lactoferrin

According to one embodiment of the invention, there is provided a glycosaminoglycan inhibitor-or promoter-composition (or a composition for inhibition or promotion of glycosaminoglycans) containing lactoferrin. In the above inhibitor or promoter composition, the lactoferrin can be iron-free (Apo) or iron saturated (Holo) lactoferrin. The lactoferrin-containing glycosaminoglycan inhibitor or promoter composition of the present invention exploits the ability of lactoferrin to inhibit or promote the expression or function of glycosaminoglycans, and the glycosaminoglycan inhibitor or promoter composition of the present invention enables the expression or function of glycosaminoglycans is inhibited or facilitated.

The glycosaminoglycan inhibitor or promoter composition of the present invention may include, in addition to lactoferrin, any solvent, dispersant, binder, additive, or other excipient known in the art.

Glycosaminoglycan Inhibitor or promoter composition containing lactoferrin of the present invention can be used in vitro or in vivo.

### 3. Application of Glycosaminoglycan Inhibitor or Promoter Composition Containing Lactoferrin in Pharmaceuticals

According to a further embodiment of the present invention, a pharmaceutical composition for treating a disease or condition related to glycosaminoglycans (hereinafter simply referred to as “the pharmaceutical composition of the present invention”) containing lactoferrin is provided. In the pharmaceutical compositions of the present invention, the lactoferrin is iron-free (Apo) or iron saturated (Holo) and is typically prepared to have an amount of lactoferrin between 0.001 and 10 g/kg/day. The subject may be a human or an animal (e.g., a mammal other than a human). The pharmaceutical compositions of the present invention can also be used in combination (concurrently or continuously) with other agents (e.g., chondroitin sulfate-degrading enzymes).

Since lactoferrin is a protein originally present in living organisms and also in foods, it has the advantage that when used as a medicine according to the present invention, it has fewer side effects, does not cause pain to the patient or the subject, and has less risk of inducing other diseases.

Examples of “diseases or conditions associated with glycosaminoglycan function” typically include, but are not limited to, cancer (e.g., lung, breast, testicular, liver, etc.), cancer metastases, diseases associated with axonal regeneration (e.g., spinal cord injury, Alzheimer's disease, etc.), diabetes, inflammation, atherosclerosis, multiple sclerosis, fibrosis, and acute respiratory disease.

In the pharmaceutical compositions of the present invention, the lactoferrin can be formulated as it is, or formulated together with a pharmaceutically acceptable carrier or the like as a medicine and administered to humans or animals for the treatment of various diseases. Accordingly, the present invention also provides a method of treating a disease or condition associated with (a function of) glycosaminoglycans, including the step of administering the lactoferrin to a patient or subject in need of treatment.

According to a further embodiment of the invention, lactoferrin can be used for the manufacture of pharmaceuticals for the treatment of diseases or conditions associated with glycosaminoglycans.

In the pharmaceutical compositions of the present invention, the lactoferrin can be provided in a kit included in said kit as one of the components of a medical (e.g., for treatment or prevention) kit. The kit may include said pharmaceutical composition as well as instructions for use describing the method of application, the amount to be applied, etc.

The term “pharmaceutically acceptable” or “pharmacologically acceptable” is used herein to describe such substances, materials, compositions, and/or dosage forms that are free from excessive toxicity, irritation, allergic reactions, or other problems or complications, are commensurate with a reasonable benefit/hazard ratio, and are within the correct medical judgment of being appropriate for use in contact with human and animal tissue.

As used herein, “pharmaceutically (or pharmacologically) acceptable carriers” include any solvent, dispersant, coating, surfactant, antioxidant, preservative (e.g., antimicrobial, antifungal), isotonic agent, absorption retarder, salt, preservative, drug, drug, drug stabilizer, gel, binder, additive, disintegrant, lubricant, sweetener, flavoring agent, dye, and/or materials and combinations thereof known in the art.

As used herein, “pharmaceutically (or pharmacologically) acceptable carriers” include any solvent, dispersant, coating, surfactant, surfactant, antioxidant, preservative (e.g., antimicrobial, antifungal), isotonic agent, absorption retarder, salt, preservative, drug, drug, drug stabilizer, gel, binder, additive, disintegrant, lubricant, sweetener, flavoring agent, dye, and/or materials and combinations thereof known in the art.

As used herein, “treatment” or “treating” includes (i) preventing a pathological condition from occurring (e.g., prevention), (ii) blocking the pathological condition or its development, (iii) reducing, remitting, or curing the pathological condition and/or reducing, remitting, or curing the pathological condition and associated symptoms.

When lactoferrin is administered to a patient or subject as a medicine according to the present invention, it can be formulated, e.g., lactoferrin alone or with customary excipients, and used for oral or parenteral administration as capsules, tablets, injections, or other appropriate dosage forms. For example, the capsules can be prepared by mixing the inhibitor or promoter composition or lactoferrin of the present invention with excipients such as lactose, starch or derivatives thereof, cellulose derivatives, etc., and filling the gelatin capsules.

Besides the above excipients, the tablets can be prepared into tablets using an ordinary compression tableting machine by adding binders such as sodium carboxymethylcellulose, alginate, gum arabic, and the like, and water to knead the tablets into granules, if necessary, and then adding a lubricant such as talc or stearic acid.

Moreover, for parenteral administration by injection, lactoferrin is dissolved in sterile distilled water or sterile saline, together with a dissolution aid, and sealed in an ampoule to make a formulation for injection. If necessary, stabilizers, buffering substances, and the like may be added. These parenteral preparations may be administered intravenously or by intravenous infusion.

Furthermore, lactoferrin may be added to nutritional supplements, food, and beverages, etc., either as is or after being formulated.

According to the present invention, the dose of lactoferrin varies according to various factors, such as symptoms, severity, age, and presence of complications of the patient or subject to be treated. It also varies depending on the route of administration, dosage form, number of doses, etc., but in general, when administered orally, the dosage of lactoferrin can usually be 0.001 to 10 g/kg/day, 0.005 to 10 g/kg/day, 0.01 to 10 g/kg/day, and 0.01 to 5 g/kg/day, as an active ingredient. When administered to humans, typically, the therapeutically effective doses are 10 mg to 15,000 mg, 10 mg to 12,000 mg, 10 mg to 10,000 mg, 20 mg to 10,000 mg, 20 mg to 8,000 mg, 30 mg to 8,000 mg, and 30 mg to 6,000 mg per day. Such a daily dose can be administered in a single or divided dose per day to patients in need of treatment of a disease or condition related to glycosaminoglycans. In the case of parenteral administration, the dose can be selected and administered in the range of about 1/100 to ½ of the dose in the case of oral administration, as appropriate.

The present invention is described in more detail by the following examples. These examples are illustrative only and do not limit the scope of the invention in any manner.

### EXAMPLES

Examples are given below. Abbreviations in the text and the figures are human lactoferrin (hLF), human lactoferrin N-lobe (hLF N-lobe), hinge deletion human lactoferrin/human IgG Fc fusion protein (hLF-CH2-CH3), human lactoferrin/human serum albumin fusion protein (hLF-HSA), human lactoferrin-human granulocyte colony-stimulating factor (G-CSF) fusion protein (hLF-G-CSF), human IgG (hIgG), human serum albumin (HSA), chondroitin sulfate A, B, C, D, E (CS-A, B, C, D, E), heparin (HP), sulfated glycosaminoglycan (sGAG) and the chick embryo spinal cord dorsal root ganglion (DRG).

### Example 1: Secondary Structure and Evaluation of Thermal Stability of Full-length Human Lactoferrin (hLf) and hLf Spherical Domains in the Presence of Various Glycosaminoglycans

The molar ratios of hLf, hLf N-lobe, and sGAG glycans were set to 1:0, 1:1, and 1:5, and the total liquid volume was prepared to 250 μl in PBS (−). The prepared samples were incubated in an aluminum block at 37° C. for 1 hour. A circular dichroism dispersion meter J-1500 (JASCO) is shown in Tables 2-1 and 2-2.

TABLE 2-1
Measurement Conditions for Secondary Structure Evaluation

| CD Scale | 200 mdeg/1.0 dOD | |
|---|---|---|
| FL Scale | 200 mdeg/1.0 dOD | |
| Response | 1 | sec |
| Band Width | 1.00 | nm |
| Starting Wavelength | 250 | nm |
| End Wavelength | 200 | nm |
| Data Capture Interval | 0.1 | nm |
| Scanning Mode | continuous | |
| Scanning Speed | 20 | nm/min |
| Stacking Times | 3 | |
| Temperature in the holder | 37° | C. |

TABLE 2-2
Measurement Conditions for Circular Dichroism Dispersion Meter

| CD scale | 200 mdeg/1.0 dOD | |
|---|---|---|
| FL Scale | 200 mdeg/1.0 dOD | |
| Response | 1 | sec |
| Bandwidth | 1.00 | nm |
| Measurement Wavelength | 225 | nm |
| Starting Temperature | 30° | C. |
| End Temperature | 90° | C. |
| Data Capture Interval | 1° | C. |

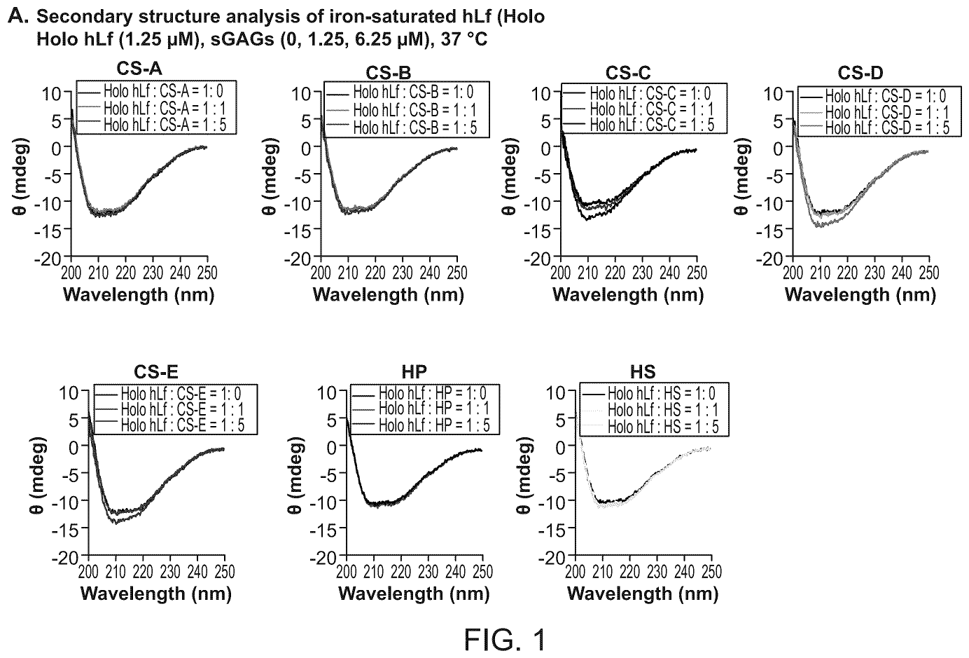

FIG. 1 shows the results of secondary structure analysis of (Apo and Holo) full-length human lactoferrin (hLf) with different degrees of iron-binding in the presence of various glycosaminoglycans by CD spectroscopy. FIG. 1A shows the results of secondary structure analysis of iron-saturated hLf (Holo hLf) and FIG. 1(B) shows the results of secondary structure analysis of iron-free hLf (Apo hLf). In the presence of CS-C, CS-D, and CS-E, a remarkable change in the secondary structure of full-length hLf was observed. This effect was not affected by the presence or absence of iron in hLf.

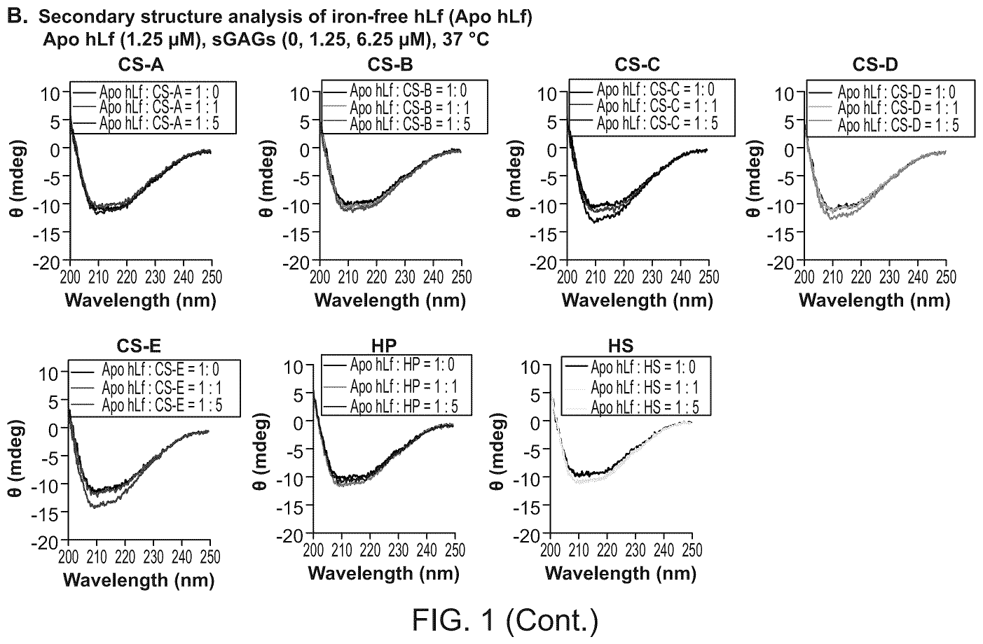

FIG. 2 shows the results of secondary structure analysis of hLf N-lobe with different degrees of iron-binding in the presence of various glycosaminoglycans by CD spectroscopy. FIG. 2A shows the results of secondary structure analysis of the iron-saturated hLf N-lobe (Holo hLf N-lobe), and FIG. 2B shows the results of secondary structure analysis of the iron-free hLf N-lobe (Apo hLf N-lobe). The results of the structural analysis show that the secondary structure of hLf N-lobe was significantly altered in the presence of CS-C, CS-D, and CS-E. This effect was not affected by the presence or absence of iron in the hLf N-lobe.

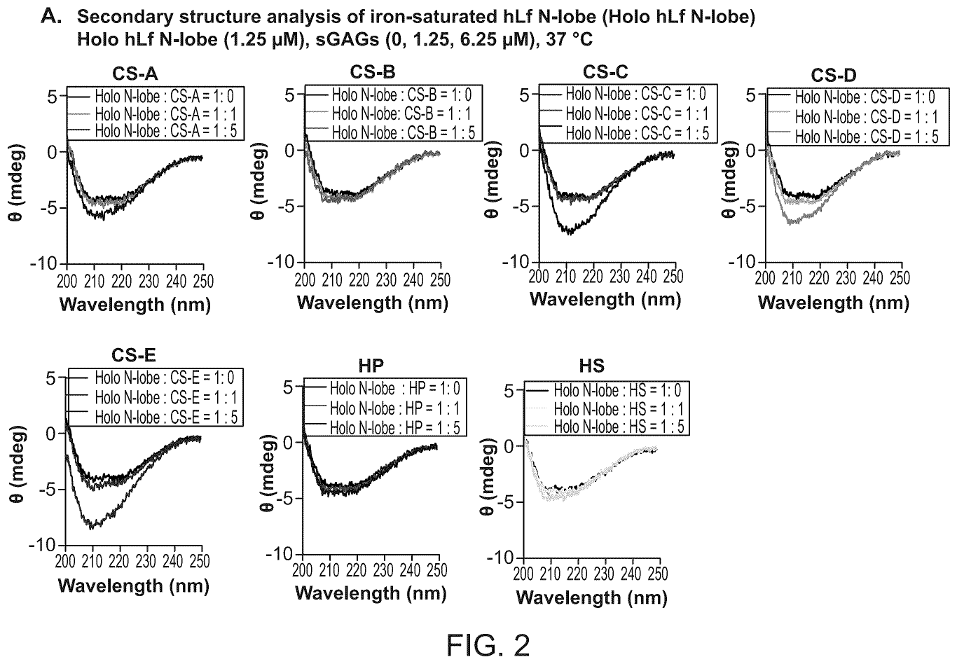

FIG. 3 shows the results of thermal denaturation studies of full-length human lactoferrin (hLf) and hLf N-lobe, respectively, with different degrees of iron-binding in the presence of various glycosaminoglycans by CD spectroscopy. FIG. 3A shows the thermal stability of Apo/Holo hLf (left) and Apo/Holo hLf N-lobe (right) in the absence of glycosaminoglycans. FIG. 3B shows the thermal stability of Apo hLf in the presence of various glycosaminoglycans (CS-A to E (left), HP, and HS (right)). FIG. 3C shows the thermal stability of Holo hLf N-lobe in the presence of various glycosaminoglycans (CS-A to E (left), HP, and HS (right)). FIG. 3D shows the thermal stability of Apo hLf N-lobe in the presence of various glycosaminoglycans (CS-A to E (left), HP, and HS (right)). The thermal stability of full-length hLf and hLf N-lobe, in the presence of CS-E, was significantly improved. This effect was not affected by the presence or absence of iron in hLf and hLf N-lobe.

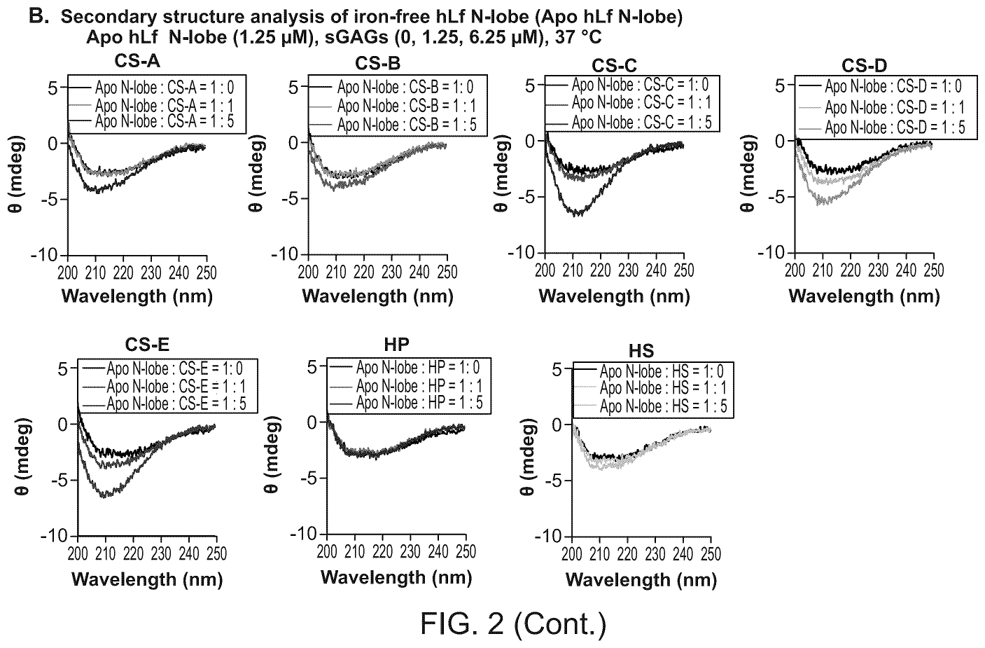

FIG. 4 shows the results of secondary structure analysis of hLfcin1-11 (GRRRRSVQWCA) in the presence of various glycosaminoglycans by CD spectroscopy. The secondary structure of hLfcin1-11 was altered in the presence of CS-C, CS-D, and CS-E.

### Example 2: Activity of Iron-Saturated Human Lactoferrin N-lobe against PC-3 Treated with Sodium Chlorate (NaClO₃)

### 2-1. Cell Proliferation Test

Sodium chlorate (NaClO₃) (Wako) was dissolved in ultrapure water to a final concentration of 1 M. The prepared solution was filter sterilized using a 0.22 μm syringe filter. Sodium chlorate treatment has been reported to inhibit the sulfation of their glycans to cells (Hoogewerf et al., The Journal of Biological Chemistry, 266, 16564-16571 (1991)). The present evaluation was used to assess whether cell surface glycans are involved in the activity of the N-lobe.

96-well plates were coated with gelatin. The gelatin solution was discarded and cells were seeded at 5×10³ cells/well in the presence of 1 mM sodium chlorate and incubated in a 37° C. 5% CO₂ incubator for 24 hours. As a control, cells seeded in the absence of sodium chlorate were prepared and cultured in the same manner. The sodium chlorate solution was adjusted using RPMI-1640 containing 5% FBS to a final concentration of 1 mM. The supernatant was discarded and replaced with a test medium: the cells were incubated in a 37° C. 5% CO₂ incubator for 3 days. 10 μl/well of Cell Counting kit-8 was added and allowed to develop color for 2 hours in a 37° C. 5% CO₂ incubator. The absorbance was measured at 450 nm using a microplate reader.

96-well plates were coated with gelatin. The gelatin solution was discarded and cells were seeded at 5×10³ cells/well in the presence of 1 mM sodium chlorate and cultured in a 37° C. 5% CO₂ incubator for 24 hours. As a control, cells seeded in the absence of sodium chlorate were prepared and cultured in the same manner. Sodium chlorate and N-lobe were adjusted with RPMI-1640 containing 5% FBS to a final concentration of 1 mM and 5 μM, respectively. Sodium chlorate was adjusted to a final concentration of 1 mM using RPMI-1640 with 5% FBS. N-lobe was adjusted using RPMI-1640 with 5% FBS to a final concentration of 5 M. RPMI-1640 containing 5% FBS was prepared as a control. The culture supernatant was discarded and 100 μl/well of test medium was added to the culture supernatant: it was incubated in a 37° C. 5% CO₂ incubator for 3 days. 10 μl/well of Cell Counting kit-8 was added and allowed to develop color for 2 hours in a 37° C. 5% CO₂ incubator. The absorbance was measured at 450 nm using a microplate reader. If the absorbance was greater than 1, dilutions were made and measurements were performed.

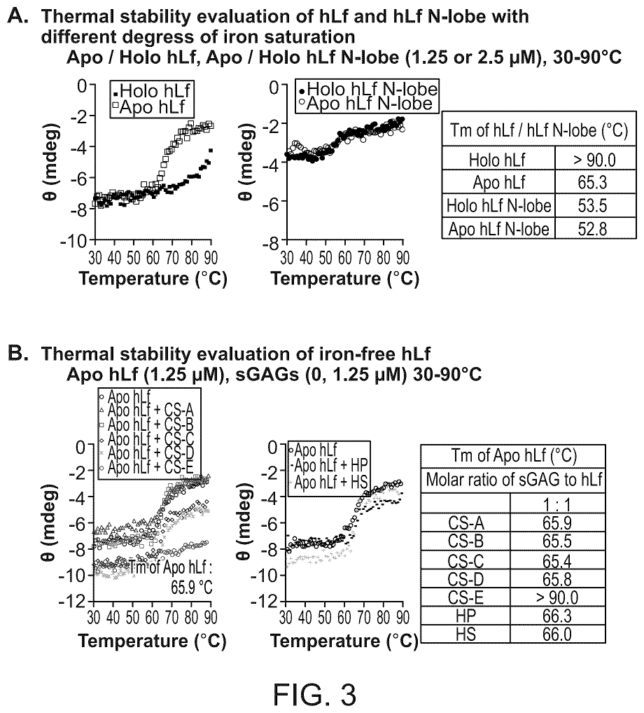

FIG. 5 shows the effect of hLf N-lobe on cell proliferation of chlorate (NaClO₃)-treated non-small cell lung cancer cell line PC-3 in the presence of various glycosaminoglycans (CS-A to E). The presence of CS-C and CS-D enhanced the inhibition of cell proliferation of hLf N-lobe on PC-3 cells, and CS-E-induced cell proliferation was inhibited by the presence of N-lobe.

### 2-2. Cell Survival Test

Glass bottom dishes (IWAKI) were coated with 0.1% gelatin. The gelatin solution was discarded and PC-3 cells were seeded at 5×10⁴ cells/dish and incubated in a 37° C. 5% CO₂ incubator for 24 h. 10 μM N-lobe solution was adjusted to 10 μM with RPMI-1640 containing 10% FBS. As a control' 0 μM, the same amount of PBS(−) was added as when 10 μM of N-lobe solution was added to the serum-containing medium. The cells were incubated in a 37° C. 5% CO₂ incubator for 3 days. 1 mg/ml PI Solution (Dojin Chemical) was added at a final concentration of 1 μg/ml, 4 mM Calcein-AM (Thermo scientific) was dissolved in RPMI-1640 at a final concentration of 400 μM to make the staining solution.

For PC-3 cells, the following procedure was followed. The supernatant was discarded and 300 μl/dish of serum-free RPMI-1640 was added and the cells were washed. The washing solution was discarded and 200 μl/dish of staining solution was added at 200 μl/dish and the cells were incubated in a 37° C. 5% CO₂ incubator for 15 minutes, while shielded from light. The supernatant was discarded and serum-free RPMI-1640 was added at 300 μl/dish and washed. For PC-3 cells, the following was performed. The supernatant was collected in 1.5 ml tubes and the cells remaining in the dish were added with 300 μl/dish of serum-free RPMI-1640. The collected supernatant was centrifuged at 200×g, 4° C. for 2 min. The supernatant was discarded and tapped with 500 μl/tube of serum-free RPMI-1640 and centrifuged at 200×g for 2 minutes at 4° C. The supernatant was discarded and 100 μl/tube of staining solution was added and tapped. The supernatant of the dish was discarded and 200 μl/dish of staining solution was added at 200 μl/dish. All cells collected in the tubes were also returned to the dish with the stain solution, and the cells were incubated in a 37° C. 5% CO₂ incubator for 15 min, while shielded from light. The supernatant was collected in 1.5 ml tubes, and serum-free RPMI-1640 was added to the remaining cells in the dish at 300 μl/dish. The collected supernatant was centrifuged at 200×g, 4° C. for 2 min. The supernatant was discarded and 1 ml/tube of serum-free RPMI-1640 was added to the supernatant and tapped. This operation was performed three times as a washing operation. The washing solution was discarded and RPMI-1640 without serum was added at 200 μl/dish. The supernatant of the dish was discarded and 200 μl/dish of serum-free RPMI-1640 was added at 200 μl/dish. The entire amount of cells collected in the tubes was also returned to the dish. The cells were observed using a scanning confocal laser microscope FV3000RS.

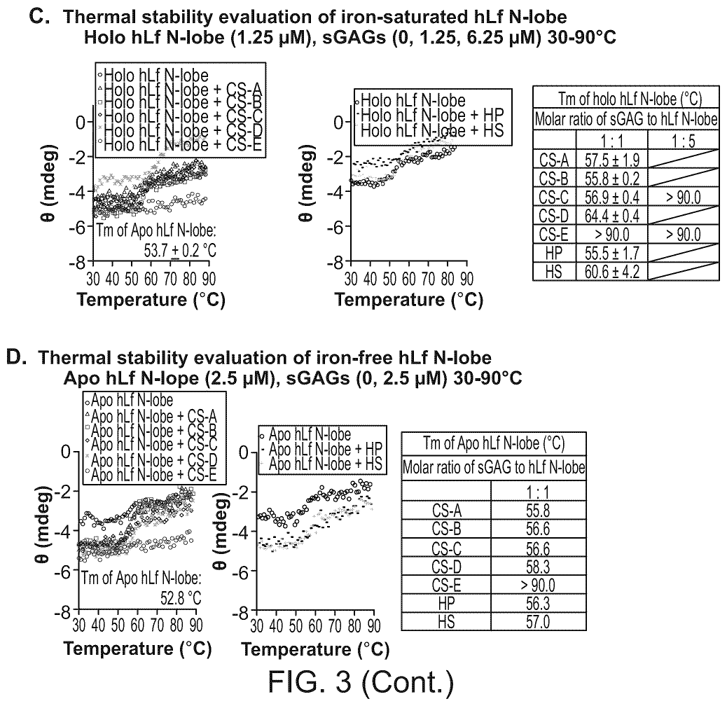

FIG. 6(A) shows the effect of iron-saturated (Holo) hLF N-lobe on cell proliferation of chlorate (NaClO₃)-treated non-small cell lung cancer cell line PC-3. A decrease in the number of viable cells and an increase in dead cells were observed with N-lobe treatment in chlorate-treated PC-3 cells, suggesting that sugars present on the cell surface attenuate the activity of the N-lobe.

### 2-3. Activity of Iron-Saturated Human Lactoferrin N-lobe on Chondroitin Sulfate Degrading Enzyme (ChABC)-Treated PC-3 Cells

PC-3 cells were seeded at 5×10³ cells/well and cultured in the presence of 1 μN/ml chondroitin sulfate ChABC (SIGMA) for 24 hours in a 5% CO₂ incubator at 37° C. As a control, cells seeded in the absence of 1 μN/ml ChABC were prepared and cultured in the same manner. When the cells were seeded, RPMI-1640 containing 5% FBS was used: RPMI-1640 containing 5% FBS, 1 μN/ml ChABC, and 5 μM N-lobe and RPMI-1640 containing 5% FBS, 1 μN/ml ChABC, was prepared. RPMI-1640 containing 5% FBS, 5 μM N-lobe, and RPMI-1640 containing 5% FBS, were also prepared. The culture supernatant was discarded and 100 μl/well of test medium was added to the culture supernatant; it was incubated in a 37° C. 5% CO₂ incubator for 3 days. 10 μl/well of Cell Counting kit-8 was added and allowed to develop color for 2 hours in a 37° C. 5% CO₂ incubator. The absorbance was measured at 450 nm using a microplate reader.

FIG. 6(B) shows the effect of iron-saturated (Holo) hLF N-lobe on the cell proliferation of chondroitin sulfate-degrading enzyme (ChABC)-treated non-small cell lung cancer cell line PC-3. In ChABC-treated PC-3 cells, the inhibition of growth by N-lobe was more enhanced, confirming that CS on the cell surface attenuates the activity of N-lobe.

Chlorate (NaClO₃) treatment alone had no particular effect on cell proliferation (FIG. 6(A)). In contrast, treatment with chondroitin sulfuric acid degrading enzyme (ChABC) alone inhibited the cell proliferation of PC-3 cells (FIG. 6(B)). This indicates that the inhibition of cell proliferation of PC-3 cells by ChABC treatment plus N-lobe was enhanced, i.e., at least an additive effect of the combination of ChABC treatment and N-lobe was confirmed.

### Example 4: Effect of the Human Lactoferrin N-Lobe on the CS-E-Dependent Metastasis of Human Lung Cancer Cells, PC-14 Cells

PC-14 cells were seeded in 12-well plates at 4×10⁵ cells/well and cultured overnight to form a cell monolayer. Uniform wounds were formed on the cell monolayer with a scratcher while applying a scratch guide. Serum-free RPMI-1640 was added at 1 ml/well and washed. The washing operation was performed three times. rhLF solution or N-lobe solution was adjusted to 5 μM with RPMI-1640 containing 10% FBS and incubated at 37° C. The culture supernatant was discarded and 400 μl/well of rhLF solution or N-lobe solution-containing medium was added. As a control, the same amount of PBS(−) medium was added at 400 μl/well as the amount of rhLF and N-lobe solutions added. The cultures were incubated overnight in a 5% CO₂ incubator at 37° C. The cells' supernatant after incubation was discarded and a 4% formaldehyde solution warmed to 37° C. was added at 350 μl/well. The cells were kept at room temperature for 20 minutes to immobilize the cells. The supernatant was discarded and 1 ml/well of PBS(−) warmed to 37° C. was added at 1 ml/well and left to stand at room temperature for 10 minutes. This operation was performed three times as a washing operation. The washing solution was discarded, and 350 μl/well of staining solution (0.2% crystal violet with 2% ethanol) was added at 350 μl/well and left to stand for 15 minutes at room temperature. The staining solution was discarded and PBS(−) was added at 1 ml/well and allowed to stand for 10 min at room temperature. This operation was performed four times as a washing operation. The stained cells were observed under a microscope. The distances of the wounds were analyzed using Image J.

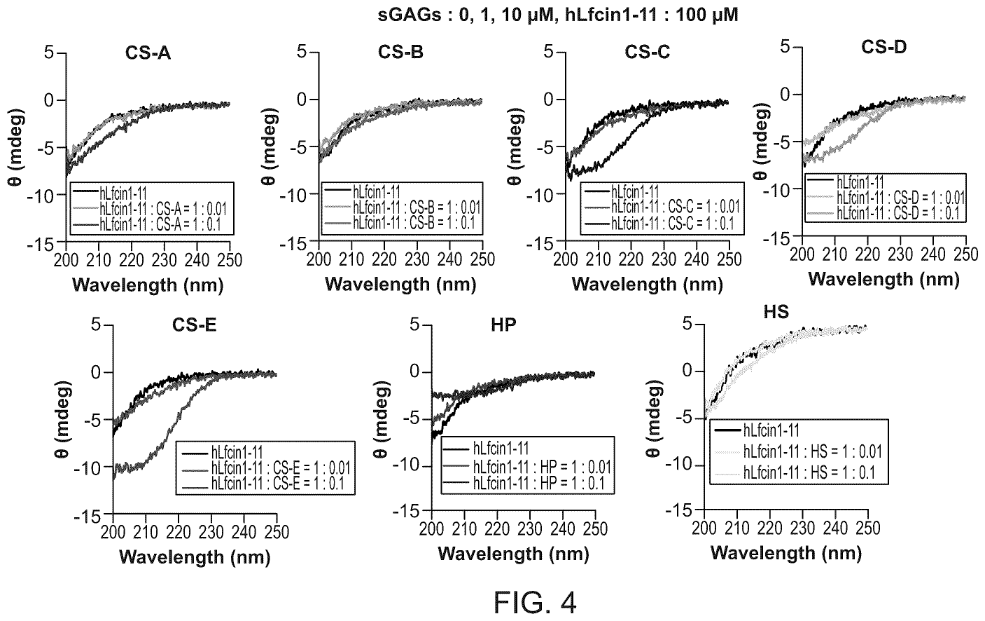

FIG. 7 shows the effect of hLF N-lobe on the CS-E-dependent metastasis of human lung cancer PC-14 cells. Evaluated using scratch assay: PC-14 cells metastasized more in the presence of CS-E (assessed by the number of cells present inside the scratch). The addition of N-lobe reduced the number of cells that metastasized in the presence of CS-E. This indicates that hLf N-lobe inhibited the CS-E-dependent metastasis of PC-14 cells.

### Example 5: Effect on Spinal Cord Injury Models Using PC-12 Cells

### 5-1. Spinal Cord Injury Model Using CS-E in PC-12 Cells

To measure the neurite length of the rat adrenal pheochromocytoma cell line PC-12 with CS glycans as a homogeneous substrate, laminin and various CS (CS-A, CS-C, CS-D, CS-E) were coated onto 12 mm glass-bottom dishes. 10 h after NGF administration, the neurite length of PC-12 cells and the number of collapsed growth cones present at the neurite tip site were calculated.

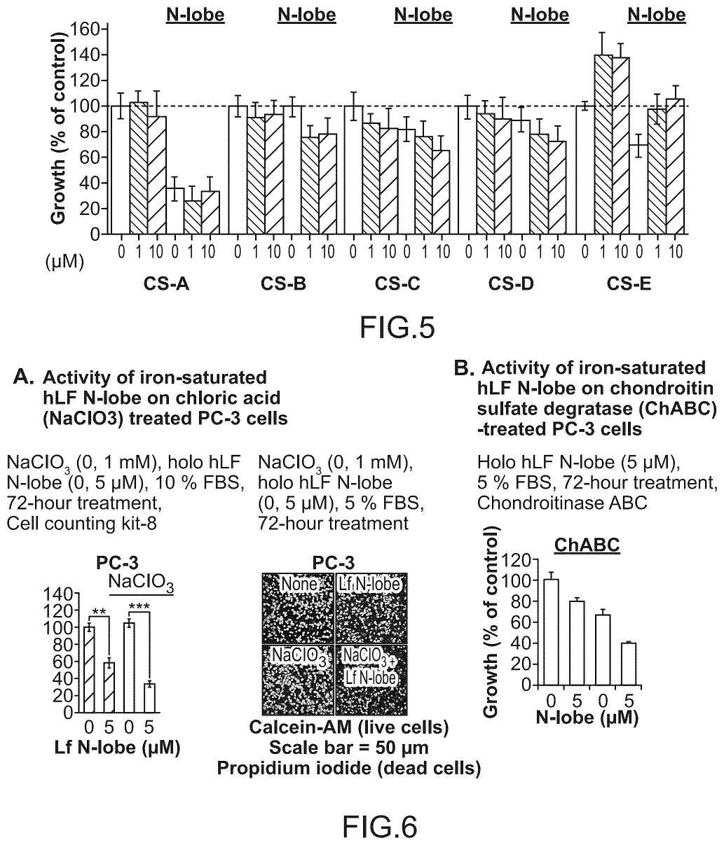

FIG. 8 shows the effects of various glycosaminoglycans (CS-A to E) on neurite outgrowth and growth cones of PC-12 cells. Axonal outgrowth was significantly inhibited in the presence of CS-E.

### 5-2. Lactoferrin N-Lobe Regulates Neurite Outgrowth by CS-E

The same moles of hLf N-lobe were added to a mixture of laminin and CS-E and the neurite length of PC-12 cells and the number of collapsed growth cones at the neurite tip after 10 hours of addition were determined.

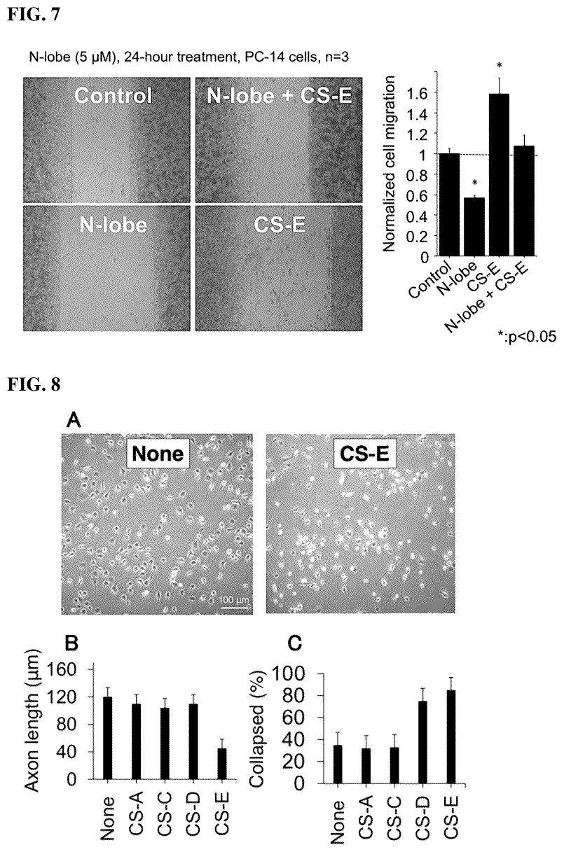

FIG. 9 shows that the inhibitory effect on outgrowth of the neurite of PC-12 cells by chondroitin sulfate E is attenuated by the administration of human lactoferrin N-lobe, which promotes neurite outgrowth.

### Example 6: Evaluation of the Binding of hLF, hLF N-lobe, and hLF Derivatives to Chondroitin Sulfate E (CS-E).

CS-E fixation plates were prepared by adding 600 nM biotinylated CS-E (PG Research) to neutral avidin-coated strip-well plates (Thermo Scientific) and allowing them to react for 2 hours at room temperature. After washing each well of the CS-E fixation plate with PBS(−), blocking was performed by shaking for 1 hour in Blocking One (Nacalitesque). Then 1.5 μM hLF, hLF N-lobe, and hLF derivatives (hLF-CH2-CH3, hLF-HSA, and hLF-G-CSF) were added and allowed to react for 4 hours. The primary antibody reaction was performed with HRP-conjugated anti-hLF antibodies (Bethyl Laboratories) diluted 1000-fold in Blocking/Sample Dilution Buffer (Chondrex) for 1 hour at room temperature. After the reaction, the reaction was washed with PBS(−) and then the chromogenic substrate solution TMB (Nacalitesque) was added and allowed to react at room temperature. The reaction was stopped with 1N HCl and the absorbance was measured at 450 nm with a microplate reader. Competitive experiments were conducted with hLF, hLF N-lobe, and hLF derivatives (hLF-CH2-CH3, hLF-HSA, hLF-G-CSF), as well as 3 μM CS-C, CS-E, HP, and HSA, and reacted for 4 hours.

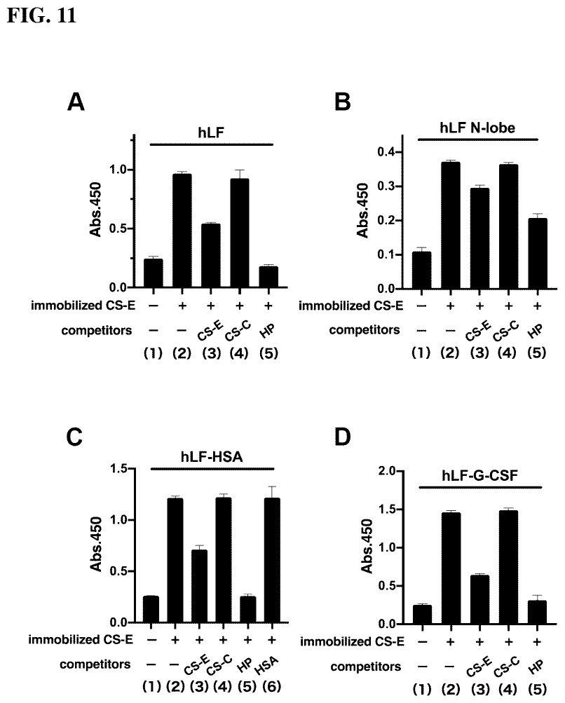

FIG. 11A shows the results of binding of hLF to sGAG (CS-E, CS-C, HP). FIG. 11B shows the results of binding of hLF N-lobe to sGAG (CS-E, CS-C, HP). FIG. 11C shows the results of binding of hLF-CH2-CH3 fusion protein (hLF-CH2-CH3) to sGAG (CS-E, CS-C, HP). FIG. 11D shows the results of binding of hLF-HSA fusion protein (hLF-HSA) to sGAG (CS-E, CS-C, HP) or human serum albumin (HSA). FIG. 11E shows the results of binding of hLF-granulocyte colony-stimulating factor (G-CSF) fusion protein to sGAG (CS-E, CS-C, HP). hLF, hLF N-lobe, and hLF derivatives (hLF-CH2-CH3, hLF-HSA, hLF-G-CSF) directly bound to CS-E (A, B, C, D, E (1) and (2)). When other sGAGs (CS-E, CS-C, HP) or HSAs were acted on the immobilized CS-E at the same time as hLF, hLF N-lobe, and hLF derivatives (hLF-CH2-CH3, hLF-HSA, hLF-G-CSF), there was competitive inhibition for CS-E and HP (A, B, C, C, D, E (3) and (5)), but not in CS-C and HSA (A, B, C, D, E (4) and C (6)).

### Example 7: Effects on Spinal Cord Injury Model Using Chick Embryo Dorsal Root Ganglion Cells

7-1. Effects of hLF, hLF Globular Domain and hLF Derivatives on Inhibition of CS-E Axon Growth

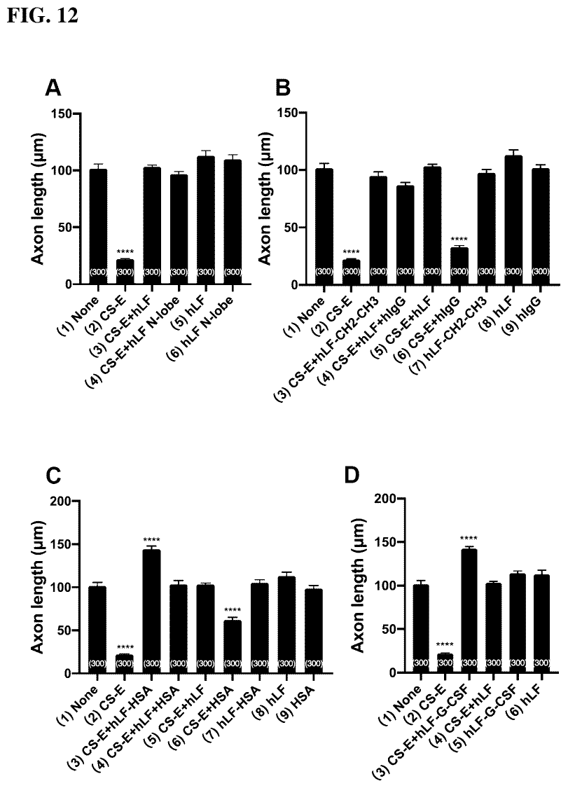

Samples of 10 μg/ml laminin (Gibco), 10 μg/ml CS-E (PG Research), and 138 nM hLF, hLF N-lobe, and a combination of hLF derivatives (hLF-CH2-CH3, hLF-HSA, and hLF-G-CSF) shown in FIG. 12, premixed and reacted for 1 hour at 37° C. were applied to a 14 mmo glass-bottom dish (Matsunami Glass Industries) coated with 100 μg/ml poly-D-lysine (Sigma-Aldrich). Spinal dorsal root ganglion cells were harvested from 8-day-old chick embryos and treated with 0.05% trypsin/EDTA at 37° C. for 18 min. DRGs were suspended in Neurobasal medium containing 2% B-27 supplement (Gibco), 1% penicillin-streptomycin solution (Thermo Fisher Scientific), and 20 ng/ml mouse NGF2.5S (Alomone Labs) and incubated at 37° C. for 24 h. Axon lengths were measured using WraySpect (Raymer). The longest axon lengths of 300 DRGs were measured and the vertical axis shows the average of the longest axon lengths.

FIG. 12A shows the results of the inhibitory effects of hLF and hLF N-lobe on the axon growth inhibitory activity of CS-E. FIG. 12B shows the results of the inhibitory effect of hLF-CH2-CH3 on the axon growth inhibitory activity of CS-E. FIG. 12C shows the results of the inhibitory effect of hLF-HSA on the axon growth inhibitory activity of CS-E. FIG. 12D shows the results of the inhibitory effect of hLF-G-CSF on the axon elongation inhibitory activity of CS-E.

The inhibitory activity of CS-E on axonal outgrowth of DRGs (A, B, C, and D (1) and (2)) was completely suppressed in the presence of hLF (A (3), B (5), C (5), and D (4)), hLF N-lobe (A (4)), hLF-CH2-CH3 (B (3)), hLF-HSA (C (3)), or hLF-G-CSF (D (3))) and the axonal outgrowth was recovered to the level of length of nerve axons (A, B, C, D (1)), which were not acted on by CS-E, or more. With the addition of hLF and hIgG (B (4)) and hLF and HSA (C (4)) as controls for the fusion proteins (hLF-CH2-CH3 and hLF-HSA), respectively, the axonal outgrowth was also recovered to the level of length of nerve axons (A, B, C (1)) that were not acted on by CS-E. In particular, hLF-HSA (C (3)) and hLF-G-CSF (D (3)) enhanced axon elongation compared to CS-E untreated nerve axons (C, D (1)), and no effect was observed when hLF and HSA were added together (C (4)), indicating effectiveness in preparing these proteins as a fusion protein. Also, for nerve axons (A, B, C, and D (1)) without CS-E action, hLF (A (5), B (8), C (8), and D (6)), hLF N-lobe (A (6)), hLF-CH2-CH3 (B (7)), hLF-HSA (C (7)), hLF-G-CSF (D (5)) did not affect axon elongation.

### 7-2. Effects of hLF and hLF Derivatives on Growth Cone Disruption of CS-E

After application of 100 μg/ml poly-D-lysine, 8-day-old chick embryo spinal cord dorsal root ganglion (DRG) cells were seeded into dishes coated with an additional 10 μg/ml laminin and then subjected to a 2% B-27 supplement (Gibco), 1% penicillin-streptomycin solution (Thermo Fisher Scientific) and 20 ng/ml mouse NGF2.5S (Promega) in Neurobasal medium (Thermo Fisher Scientific) culture medium for 24 hours. 10 μg/ml CS-E or 10 μg/ml CS-E coexisted with 138 nM hLF, hLF N-lobe, and hLF derivatives (hLF-CH2-CH3, hLF-HSA, and hLF-G-CSF) in the culture medium, and the decay rate of the growth cones was calculated after 30 minutes. The vertical axis indicates the decay rate in the total growth cones.

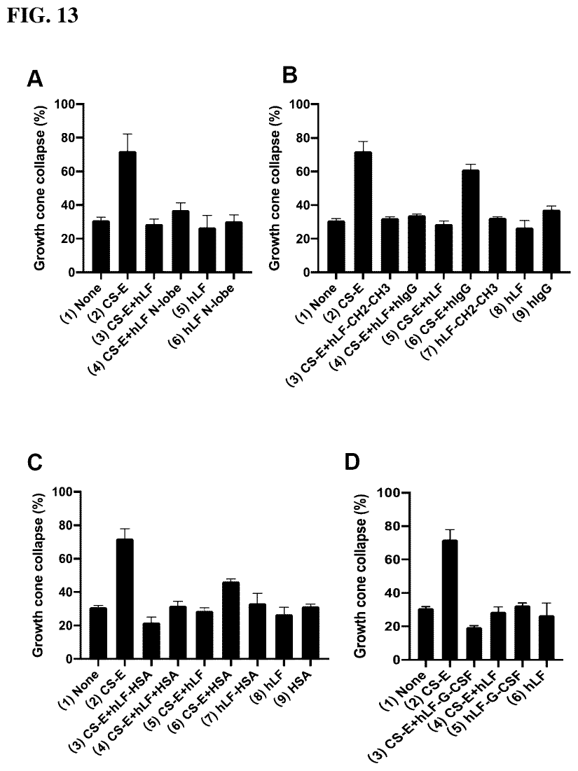

FIG. 13A shows the results of the inhibitory effect of hLF and hLF N-lobe on the disruption activity of growth cones by CS-E. FIG. 13B shows the results of the inhibitory action of hLF-CH2-CH3 on the disruption activity of growth cones by CS-E.

FIG. 13C shows the result of the inhibitory action of hLF-HSA on the disruption activity of growth cones by CS-E. FIG. 13D shows the results of the inhibitory action of hLF-G-CSF on the disruption activity of growth cones by CS-E.

Disruption activity of DRG growth cones by CS-E (A, B, C, and D (1) and (2)) was completely inhibited in the presence of hLF (A (3), B (5), C (5), and D (4)), hLF N-lobe (A (4)), hLF-CH2-CH3 (B (3)), hLF-HSA (C (3)), hLF-G-CSF (D (3))) and recovered to the same extent as the growth cone disruption (A, B, C, and D (1)) in neurons that had not been subjected to CS-E. When hLF and hIgG (B (4)) or hLF and HSA (C (4)) were added together as a control for the fusion proteins (hLF-CH2-CH3 and hLF-HSA), they also recovered to the same extent as the non-CS-E-activated neuronal growth cone disruption (A, B, C (1)). The addition of hLF-HSA (C (3)) and hLF-G-CSF (D (3)) in particular tended to lower the growth cone disruption rate than that of the untreated neurons (C, D (1). In contrast, the effect of preparing them as fusion proteins was shown. Also, neurons (A, B, C, and D (1)) without CS-E (A, B, C, and D (1)) were treated with hLF (A (5), B (8), C (8), and D (6)), hLF N-lobe (A (6)), hLF-CH2-CH3 (B (7)), hLF-HSA (C (7)), hLF-G-CSF (D (5)), respectively, did not affect the growth cone collapse.

### INDUSTRIAL APPLICABILITY

Glycosaminoglycan inhibition/promotion formulations containing lactoferrin or derivatives thereof may be used as a therapeutic agent or in the manufacture of therapeutic agents for the treatment of diseases or conditions related to glycosaminoglycan function.

1-19. (canceled)

20. A method for treating a glycosaminoglycan-related disease or condition, comprising administering lactoferrin or a derivative thereof to a patient or subject in need of treatment.

21. The method according to claim 20, wherein the lactoferrin is iron free (Apo) or iron saturated (Holo) lactoferrin, prepared in an amount from 0.001 to 10 g/kg/day.

22. The method according to claim 20, wherein the lactoferrin is of human origin.

23. The method according to claim 20, wherein the lactoferrin is a protein or peptide selected from the group consisting of (a) to (d) below:
(a) a protein consisting of any one amino acid sequence of SEQ ID NOS: 1-6;
(b) a protein comprising N-lobe of lactoferrin;
(c) a protein consisting of the amino acid sequence in which 1 to 70 amino acids are deleted, substituted, inserted, and/or added in any one amino acid sequence of SEQ ID NOS: 1 to 6, and which has activity to inhibit or promote the function of glycosaminoglycans; and
(d) a protein consisting of the amino acid sequence having 90% or more sequence identity to any one amino acid sequence of SEQ ID NOS: 1-6 and having activity that inhibits or promotes the expression or function of chondroitin sulfate.

24. The method according to claim 20, wherein the derivative of the lactoferrin is a hinge deletion human lactoferrin/human IgG Fc fusion protein (hLF-CH2-CH3) (SEQ ID NO: 9), human lactoferrin/human serum albumin fusion protein (hLF-HSA) (SEQ ID NO: 10) or human lactoferrin-human granulocyte colony-stimulating factor (G-CSF) fusion protein (hLF-G-CSF) (SEQ ID NO: 11).

25. The method according to claim 20, wherein the glycosaminoglycan is chondroitin sulfate-C (CS-C), chondroitin sulfate-D (CS-D), or chondroitin sulfate-E (CS-E).

26. The method according to claim 20, for use in combination with chondroitin sulfate-degrading enzymes.

27. The method according to claim 20, wherein the disease is any one selected from the group consisting of spinal cord injury, cancer metastasis, cancer, diabetes, inflammation, atherosclerosis, multiple sclerosis, fibrosis, acute respiratory disease, and Alzheimer's disease.

28. The method according to claim 20, which the administration is made in the form of an injection.

29. The method according to claim 20, wherein the administration is for an oral administration.
